(19) United States
(12) Patent Application Publication
(10) Pub. No.: US 2004/0063616 A1
(43) Pub. Date: Apr. 1, 2004

(54) Compositions containing peptide copper complexes and soft tissue fillers, and methods related thereto

(75) Inventor: Leonard M. Patt, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: ProCyte Corporation, Redmond, WA

(21) Appl. No.: 10/607,575

(22) Filed: Jun. 26, 2003

Related U.S. Application Data
(60) Provisional application No. 60/393,563, filed on Jul. 2, 2002.

(51) Int. Cl. A61K 38/16

(52) U.S. Cl. 514/6; 514/12; 514/54

(57) ABSTRACT

Novel compositions for treating skin defects and effecting desired cosmetic changes by way of soft tissue augmentation, combine at least one soft tissue filler and at least one peptide copper complex. Typically, the compositions are suitable for injection into skin areas in need of such treatment. Also disclosed are methods for treating skin defects and effecting desired cosmetic changes. One disclosed method employs the disclosed compositions wherein the soft tissue fillers and peptide copper complexes are combined. Other disclosed methods combine the soft tissue fillers and peptide copper complexes during application of the method itself by way of injection, or a combination of injection and topical application, of the fillers and complexes.

### CROSS-REFERENCE TO RELATED APPLICATION

This application claims the benefit of U.S. Provisional Patent Application No. 60/393,563 filed Jul. 2, 2002, which application is incorporated herein by reference in its entirety.

### BACKGROUND OF THE INVENTION

1. Field of the Invention

The present invention generally relates to compositions used for treating skin defects and/or effecting desired cosmetic changes, and, more particularly, to compositions and preparations comprising peptide copper complexes and soft tissue fillers.

2. Description of the Related Art

Soft tissue augmentation involves procedures for correcting skin defects that include injecting, immediately under the affected skin, solid or semi-solid material to fill in the defect. Defects that can be corrected this way include wrinkles caused by normal aging of the skin, depressed lines or furrows around the eye or mouth, chin and neck folds, depressions resulting from rhinoplasty, or defects associated with clinical processes, such as sunken scars resulting from acne vulgaris. Soft tissue augmentation may be more purely cosmetic in nature and involve, for example, a procedure to change the profile of the lips.

There are a number of materials that have been used for soft tissue augmentation. Some of these soft tissue fillers have been derived from cadaver or donor sources (primarily from the skin) or are synthetic polymers. Soft tissue fillers, derived from cadaver or donor tissue, typically are highly processed forms of collagen and other materials isolated from skin, autologous fat, or hyaluronic acid isolated from skin or an animal source such as rooster comb. More recently, modified hyaluronic acid produced from fermentation of genetically altered microorganisms has been used. Synthetic soft tissue augmentation products include a wide variety of materials including low melting point paraffin, vegetable oil, lanolin, beeswax, various silicon polymers, expanded polyfluoroethylene (Teflon®), polylactic and polyglutamic acid, cellulose polymers, and polymethyl methacrylate and related polymers. These soft tissue fillers are prepared in a variety for forms depending on the nature of the material and the intended use. Such forms include thick solutions, gels, microbeads, crushed beads, and suspensions, among others.

One of the drawbacks of existing soft tissue filler compositions is the need to repeat injections and applications of the compositions as the body degrades them. Such degradation typically necessitates replacement injections about every three months. Another drawback, particularly of synthetic soft tissue fillers, is their feeling different than normal tissue and their being palpable under the skin. Another problem with synthetic soft tissue fillers is their lack of biocompatibility. The latter can result in inflammatory reactions, the formation of foreign body granulomas, and encapsulation of the injected material. In some cases, these immunologically-based reactions result in over correction of the original defect resulting in a poor cosmetic outcome and additional treatment (see, e.g., Cheng, Jacqueline T., Perkins, Stephen W., and Hamilton, Mark M., “Collagen and Injectable Fillers,” Otolaryngologic Clincs of North America 35(1): 73-85, 2002; Ellis David A. F., Makdessian, Ara S., and Brown, Deron J., “Survey of Future Injectables” Facial Plastic Surgery Clinics of North America 9(3): 405-411, 2001; Maas Corey S. and Denton, Andrew B., “Synthetic Soft Tissue Substitutes,” Facial Plastic Surgery Clinics of North America 9(2): 219-227, 2001).

Accordingly, there remains a need in the art for compositions that are useful for soft tissue augmentation, while avoiding some or all of the above-described drawbacks and problems. There also remains a need in the art for methods of treating skin defects that employ such compositions. The present invention fulfills these needs and provides further related advantages.

### BRIEF SUMMARY OF THE INVENTION

In brief, the present invention is directed to compositions comprising a soft tissue filler and to methods for treating skin defects utilizing the same.

In one representative embodiment, the present invention is directed to compositions that combine at least one soft tissue filler and at least one peptide copper complex. As such compositions are useful for soft tissue augmentation, they are in a form suitable for injection under the skin in areas in need of such augmentation. In another representative embodiment, the composition comprises at least one soft tissue filler and at least one peptide copper complex, wherein the at least one peptide copper complex is encapsulated in a liposome or microsponge adapted to aid in the delivery of the complex or to enhance the stability of the composition.

Additional embodiments of the composition of the present invention further include an inert carrier or diluent, an excipient, a thickening agent (textural modifier), an emulsifying agent, a preserving agent, or mixtures thereof. These compositions may be in the form of a solution, suspension, or a gel. Pharmaceutical preparations for treating skin defects, made from these compositions, are also disclosed.

The present invention is also directed, in another representative embodiment, to a method for treating skin defects by injecting into an area of skin in need of such treatment an effective amount of a composition of the present invention. In another related embodiment, the area of skin is first injected with a composition comprising a soft tissue filler, and, then, further treated by injecting or topically applying a composition comprising a peptide copper complex in a suitable vehicle.

These and other aspects of this invention will be evident upon reference to the following detailed description of the invention.

### DETAILED DESCRIPTION OF THE INVENTION

As noted above, in one representative embodiment, there is disclosed a composition that combines at least one soft tissue filler and at least one peptide copper complex. Such compositions are in a form suitable for injection, and thus useful for soft tissue augmentation. Methods for treating skin defects and effecting desired cosmetic changes are also disclosed.

As used herein, the expressions “soft tissue augmentation” means a procedure that includes injecting a composition into an area under affected skin and/or topically applying the same or a different composition onto the affected skin, for the purpose of effecting a desired cosmetic change or correcting a skin defect. Examples of such skin defects include, but are not limited to: wrinkles, depressed lines or furrows, chin and neck folds, depressions resulting from rhinoplasty, and defects resulting from clinical processes, such as sunken scars resulting from acne vulgaris.

Accordingly, the term “injectable,” as used herein in the context of compositions useful for soft tissue augmentation, refers to compositions that can be injected into areas under affected skin to, thereby, correct skin defects, such as those listed above, as well as to correct more purely cosmetic defects, such as an undesirable lip profile, or to effect any desired cosmetic change.

The expression “soft tissue filler,” as used herein, means any solid, semi-solid, or fluid material, natural or synthetic, that can be used for soft tissue augmentation. Examples of natural soft tissue fillers include, but are not limited to, highly processed forms of collagen and other materials isolated from skin, autologous fat, hyaluronic acid isolated from skin or an animal source, and modified hyaluronic acid produced from fermentation of genetically altered microorganisms. Examples of synthetic soft tissue fillers include, but are not limited to, low melting point paraffin, vegetable oil, lanolin, beeswax, various silicon polymers, expanded polyfluoroethylene (Teflon®), polylactic and polyglutamic acid, cellulose polymers, and polymethyl methacrylate and related polymers.

Also, as used herein, the term “peptide copper complex” refers to a coordination compound comprising a peptide molecule and a copper ion non-covalently complexed therewith. The peptide molecule serves as the complexing agent by donating electrons to the copper ion to yield the non-covalent complex. The peptide molecule is a chain of two or more amino acid units covalently bonded together via amide linkages (for example, —CONH—), the formation of such linkages being accompanied by the elimination of water. The amino acid units are from amino acids that are naturally occurring or otherwise. Also, at least one amide linkage nitrogen atom may have covalently bonded thereto either a hydrogen atom or another moiety.

Generally, an amino acid consists of an amino group, a carboxyl group, a hydrogen atom, and an amino acid side-chain moiety—all bonded, in the case of an alpha-amino acid, to a single carbon atom that is referred to as an alpha-carbon. The amino acid units of the peptide copper complexes comprised in compositions of the present invention may be provided by amino acids other than alpha-amino acids. For example, the amino acids may be beta- or gamma-amino acids, such as those shown below.

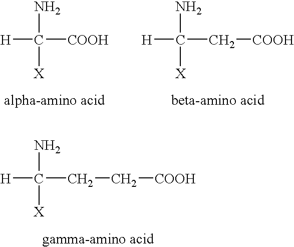

where X is the amino acid side-chain moiety.

Naturally occurring amino acids, that is, amino acids from which the amino acid units of naturally occurring proteins are derived, and their respective naturally occurring, amino acid side chain moieties, are shown below in Table 1. These naturally occurring amino acids are all in the L configuration, referring to the optical orientation of the alpha carbon or other carbon atom bearing the amino acid side chain. The amino acids comprising the peptide molecule can also be of the D optical configuration.

TABLE 1
NATURALLY OCCURRING AMINO ACID SIDE-CHAIN MOIETIES

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 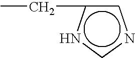 | Histidine |
| —CH₂COO— | Aspartic Acid |
| —CH₂CH₂COO— | Glutamic Acid |
| —CH₂CONH₂ | Asparagine |
| —CH₂CH₂CONH₂ | Glutamine |
| 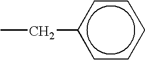 | Phenylalanine |
| 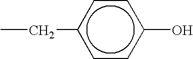 | Tyrosine |
| 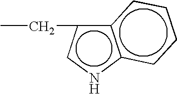 | Tryptophan |
| —CH₂SH | Cysteine |
| —CH₂CH₂SCH₃ | Methionine |
| —CH₂OH | Serine |
| —CH(OH)CH₃ | Threonine |

One example of a copper peptide complex is alanyl-histidyl-lysine:copper(II). Copper(II), as is well understood by the skilled artisan, designates a copper ion having a valence of 2 (e.g., Cu⁺²). Additional examples of the peptide copper complexes, encompassed in embodiments of the present invention, include, but are not limited to, those described in U.S. Pat. Nos. 4,665,054; 4,760,051; 4,767,753; 4,877,770; 5,023,237; 5,059,588; 5,120,831; 5,135,913; 5,145,838; 5,177,061; 5,214,032; 5,348,943; 5,538,945 and 5,550,183, incorporated herein by reference in their entireties.

Further, the expression “peptide copper complex,” as used herein, encompasses peptide copper complex derivatives. The expression “peptide copper complex derivative,” as used herein, refers to a peptide copper complex where the peptide molecule thereof has: 1) at least one amino acid side chain moiety that is a modification and/or variation of a naturally occurring, amino acid side-chain moiety; and/or 2) at least one of the hydrogens, bonded to an amide linkage nitrogen atom, substituted with a different moiety; and/or 3) the carboxyl group of the carboxyl terminal residue esterified or otherwise modified; and/or 4) at least one hydrogen, bonded to the nitrogen atom of the amino-terminal residue, substituted with a different moiety.

For example, the amino acid side-chain moieties of alanine, valine, leucine, isoleucine and phenylalanine may generally be classified as lower chain alkyl (1-12 carbon atoms), lower chain aryl (6-12 carbon atoms), or lower chain aralkyl (7-12 carbon atoms) moieties. The amino acid side-chain moieties of the peptide copper complex derivatives, may include other straight chain or branched, cyclic or noncyclic, substituted or unsubstituted, saturated or unsaturated lower chain alkyl, aryl or aralkyl moieties. Also, the peptide copper complex derivative may, for example, be N-alkylated at one or more peptide bonds; and/or its carboxyl terminus may be esterified, for example, with a methyl, ethyl, or benzyl group, or may be reduced to a hydroxy or aldehyde. Additionally, the peptide copper complex derivative may, for example, be N-alkylated, N-acylated or N-sulfonylated at the amino terminus with, for example, methyl, benzyl, acetyl, benzoyl, methanesulfonyl, or fluorenyloxycarbonyl moieties.

Examples of the peptide copper complex derivatives, encompassed in embodiments of the present invention, include, but are not limited to, those disclosed and described in the above-cited U.S. Patents that are directed to peptide copper complexes, as well as those disclosed and described in the published PCT application having the international publication number WO 94/03482, incorporated herein by reference in its entirety.

Copper is known to have many beneficial biological applications, including stimulating a variety of processes related to skin, for example, collagen, elastin and glycosaminoglycan production (see, e.g., Maquart, F. X., Pickart, L., Laurent, M., Gillery, P., Monboisse, J. C., Borel, J. P., “Stimulation of Collagen Synthesis in Fibroblast Cultures by the Tripeptide-Copper Complex Glycyl-L-Histidyl-L-Lysine-Copper(2+),” FEBS Lett. 238(2): 343-346, 1988; Wegrowski, Y., Maquart, F. X. and Borel, J. P., “Stimulation of Sulfated Glycosaminoglycan Synthesis by the Tripeptide-Copper Complex Glycyl-L-Histidyl-L-Lysine-Copper(2+),” Life Sciences 51: 1049-1056, 1992; Maguart, F. X., Bellon, G., Chaqour, B., Wegrowski, J., Patt L. M., Trachy, R. E., Monboisse, J. C., Chastang, F., Birembaut, P., Gillery, P. and Borel, J. P., “In Vivo Stimulation of Connective Tissue Accumulation by the Tripeptide-Copper Complex Glycyl-L-Histidyl-L-Lysine-Copper(2+) in Rat Experimental Wounds,” J. Clin. Invest. 92: 2368-2376, 1993). The above-cited references are incorporated herein by reference in their entireties.

Copper salts alone are ineffective, or even inhibitory, for such applications. The copper must be delivered in a biologically acceptable form. As an example, when copper is complexed with a biologically acceptable carrier molecule, such as a peptide, it may then be effectively delivered to cells.

The ability of peptide copper complexes to increase the amount of collagen in skin and to stimulate natural extracellular matrix accumulation by, for example, stimulating the accumulation of collagen, elastin and glycosaminoglycan, is of particular relevance to the present invention. More specifically, this ability underlies the use of peptide copper complexes, in combination with soft tissue fillers, to mitigate or eliminate the above-described drawbacks and problems associated with using soft tissue fillers for treating skin defects and effecting desired cosmetic changes through soft tissue augmentation. In combining soft tissue fillers and peptide copper complexes for such applications, the soft tissue filler is used to provide immediate correction of the defect, while the peptide copper complex is used to correct the skin defect for the long term. Advantages of this approach include reducing the frequency of repeat treatments and injecting less material per treatment during the course of treatments to eliminate the skin defect.

In certain specific embodiments of the composition of the present invention, the at least one peptide copper complex is alanyl-histidyl-lysine:copper(II) (“AHK-Cu”), valyl-histidyl-lysine:copper(II) (“VHK-Cu”), or glycyl-histidyl-lysine:copper(II) (GHK-Cu”), respectively. As is well understood in the art, copper(II) designates a copper ion having a valence of 2 (e.g., Cu⁺²). Further, such peptides may be in either the L or D form. In a related, more specific embodiment, they are all in the L form.

In another specific embodiment, the composition of the present invention includes the peptide copper complex derivative that is a derivative of GHK-Cu having the general formula:

[glycyl-histidyl-lysine-R]:copper(II)

where R is an alkyl moiety containing from one to eighteen carbon atoms, an aryl moiety containing from six to twelve carbon atoms, an alkoxy moiety containing from one to twelve carbon atoms, or an aryloxy moiety containing from six to twelve carbon atoms. This derivative of GHK-Cu is further described in the above-cited U.S. Patents that are directed to peptide copper complexes.

Compositions of the present invention, in further related embodiments, comprise peptide copper complexes where the molar ratio of peptide to copper in the peptide copper complex ranges from about 1:1 to about 3:1, and from about 1:1 to about 2:1, respectively, and where the concentration of the peptide copper complex ranges from about 0.01% to about 10%, from about 0.025% to about 1%, and from about 0.05% to about 0.5%, respectively, based on the weight of the composition.

In another specific embodiment directed to compositions, the at least one soft tissue filler is a natural material derived from animal tissue. In more specific, related embodiments, the natural material is collagen, autologous fat, or hyaluronic acid, including a modified form thereof. In yet another specific embodiment directed to compositions of the present invention, the at least one soft tissue filler is a synthetic material which, in further, more specific related embodiments, is a low melting point paraffin, a vegetable oil, lanolin, beeswax, a silicon polymer, expanded polyfluoroethylene (Teflon®), polylactic acid, polyglutamic acid, a cellulose polymer, and polymethyl methacrylate, or a polymer based on polymethyl methacrylate.

The concentration of the soft tissue filler, in certain embodiments, ranges from about 0.001% to about 99%, from about 0.01% to about 90%, and from about 0.01% to about 50%, respectively, based on the weight of the composition.

The disclosed compositions may be prepared by combining soft tissue fillers, prepared as gels or fine suspensions, and aqueous solutions of peptide copper complexes. Such gels and fine suspensions are prepared by methods that are well known to those skilled in the art. Further, such aqueous solutions are also prepared by methods that are well known to those skilled in the art. For example, an amount of dried peptide copper complex suitable for a desired concentration is readily dissolved in water with mixing and gentle heating. An alternative method is to prepare a solution of the desired peptide, followed by the addition of a copper salt in the desired molar ratio to yield the desired solution of the peptide copper complex. Examples of copper salts that may be used are cupric chloride and cupric acetate. When aqueous solutions of peptide copper complexes are prepared, the solutions are neutralized, typically with NaOH.

The present invention, in another representative embodiment, is also directed to an injectable soft tissue augmentation composition formed by combining at least one peptide copper complex with at least one soft tissue filler, where the combined compounds or the peptide copper complex is encapsulated in liposomes or microsponges to aid in the delivery of the peptide copper complex or to increase the stability of the composition.

The compositions of the present invention are intended primarily as products for injection into human skin. Accordingly, in a particular embodiment, the compositions are in the form of a solution, thick solution, suspension, or gel. Also, in another particular embodiment, the compositions, and preparations comprising the compositions, further comprise suitable excipients adapted for injection into skin. Suitable excipients should be well tolerated, stable, and yield a consistency that allows for easy and pleasant utilization.

In yet other particular embodiments, the compositions of the present invention, and preparations derived therefrom, further comprise an additional agent, such as: an inert and physiologically-acceptable carrier or diluent, an excipient, a thickening agent (textural modifier), an emulsifying agent, a preservative, and a mixture thereof, respectively. Suitable examples of the above additional agents typically include those agents commonly used in pharmaceutical and skin care preparations. More specifically, such examples of an inert and physiologically-acceptable carrier or diluent include saline and purified water. Such examples of an excipient include phosphate buffered saline, bacteriostatic saline, propylene glycol, starch, sucrose and sorbitol. Suitable thickening agents include acrylamides copolymer, carbomer, hydroxyethylcellulose, hydroxypropylcellulose, polyacrylic acid, polymethacrylic acid and polyvinyl alcohol.

Suitable emulsifying agents include caprylic/capric triglyceride, ceteareth-7, cetyl alcohol, cetyl phosphate, isosteareth-11 and sodium isostearate. Preservatives impart to the compositions of the present invention, resistance to microbial attack and toxicity to microbes. Suitable examples include benzyl alcohol, any of the parabens, diazolidinyl urea, DMDM hydantoin, phenoxyethanol, and iodopropynyl butylcarbamate. Examples of the above additional agents, other than those that are listed, may also be used in embodiments of this invention, as would be well appreciated by one of ordinary skill in the art.

In another aspect, the present invention is directed to a method for treating skin defects and effecting more purely cosmetic changes, examples of such skin defects and cosmetic changes including those listed previously. In one such embodiment, the method comprises the step of injecting into an area of skin in need of such treatment, a composition of the present invention that combines at least one soft tissue filler and at least one copper peptide complex. In another such embodiment, the method comprises injecting into an area of skin in need of such treatment, an effective amount of a soft tissue filler, followed by injecting into the area an effective amount of a peptide copper complex.

In yet another related embodiment, the method comprises injecting into an area of skin in need of such treatment, an effective amount of a soft tissue filler, followed by topically applying an effective amount of a peptide copper complex. The peptide copper complex-containing composition that is topically applied in the latter method, in addition to comprising an additional agent, such as those previously described, may also further comprise a sunscreen agent, a skin lightening agent, a tanning agent, a skin conditioning agent, a skin protectant, an emollient, a humectant, or a mixture thereof.

The following examples are provided for the purpose of illustration, not limitation.

### EXAMPLES

### Example 1

### Stimulation of Collagen and Glycosaminoglycan Synthesis by Injection of a Representative Soft Tissue Filler and a Representative Peptide Copper Complex

The subcutaneous implantation of stainless steel chambers in rats provides a model for studying the synthesis of extracellular matrix components (collagen and glycosaminoglycan) by providing a recoverable site of new matrix synthesis. The assay involves implanting in each rat, two cylindrical stainless steel chambers (1 cm in diameter×2.5 cm long, 312 SS, 20 mesh, with Teflon end caps), one on each side of the rats' dorsal midline. After allowing for encapsulation of the chambers, both chambers on each rat were injected with 0.2 ml of a solution containing the representative soft tissue filler or saline on day 4 after implantation, and the test peptide copper compound (or saline alone as a control) on days 6, 8, 11, 13, 15, 16, and 18. Chambers were removed from the animals on day 30 after implantation for biochemical analysis.

The chambers were lyophylized and the interior contents removed for biochemical analysis. The biochemical parameters examined include collagen content, the latter being measured as a hydroxyproline (“HYP”) content. The latter, an amino acid specific for collagen, was measured after acid hydrolysis and using a colormetric assay for HYP (see e.g., Bergman, I and Loxley, R., “The Determination of Hydroxyproline in Urine Hydrolysates,” Clin. Chim. Acta. 27: 347-349, 1970). Collagen content was expressed as μg of HYP per chamber or per milligram of protein.

The chambers were also analyzed for glycosaminoglycan content, another component of the extracellular matrix or skin. Glycosaminoglycan (“GAG”) content was determined by quantifying the amount of uronic acid (“UA”), a carbohydrate component specific for GAGs. UA was determined by a colorimetric assay, as described using 2-hydroxydiphenyl as a reagent (see, e.g., Vilim, V., “Colorimetric Estimation of Uronic Acids using 2-hydroxydiphenyl as a Reagent,” Biomed. Biochim. Acta. 44 11/12 s: 1717-1720, 1985). GAG content was expressed as μg of UA per chamber.

In this example, hydroxypropyl methyl cellulose was used as the soft tissue filler. A dose of 6 milligrams of the hydroxypropyl methyl cellulose, as a gel, was injected into each of the chambers. Injections of saline served as controls.

Glycyl-L-histidyl-L-lysine:copper(II) (“GHK-Cu”), was used as the peptide copper complex used. The GHK-Cu was prepared at a molar ratio of 2 moles of peptide to one mole of copper(II), and dissolved in a saline solution at a concentration of 10 milligrams/milliliter. A dose of 0.2 mg of GHK-Cu was injected for each day of treatment.

The results of injecting GHK-Cu as the peptide copper complex and hydroxypropyl methylcellulose (HPMC) as the soft tissue filler for stimulating collagen formation are shown in the table below for 4 groups of rats. Group 1 rats were the control rats injected with saline only. Group 2 rats were injected with only the peptide copper complex (GHK-Cu) solution. Group 3 rats were injected with the tissue filler (HPMC) only. Group 4 rats were injected with both the tissue filler (HPMC) and the peptide copper complex (GHK-Cu).

| | Soft Tissue | Peptide | μg HYP/Chamber |
|---|---|---|---|
| Group | Filler | Copper Complex | Mean ± SEM |
| 1 | − | − | 1222 ± 111 |
| 2 | − | + | 4226 ± 265 |
| 3 | + | − | 1506 ± 151 |
| 4 | + | + | 3423 ± 341 |

As is evident from the results shown in the above table, injecting the tissue filler, alone, does not result in any stimulation of collagen synthesis, while injecting the peptide copper complex, either alone or in combination with the tissue filler, does result in an increase in collagen synthesis.

The results of injecting GHK-Cu as the peptide copper complex and hydroxypropyl methyl cellulose as the soft tissue filler for stimulating GAG (as UA) formation are shown in the table below for 4 groups of rats.

| | Soft Tissue | Peptide | μg Uronic Acid/Chamber |
|---|---|---|---|
| Group | Filler | Copper Complex | Mean ± SEM |
| 1 | − | − | 46.3 ± 5.8 |
| 2 | − | + | 117.3 ± 12.9 |
| 3 | + | − | 49.9 ± 4.1 |
| 4 | + | + | 88.1 ± 5.6 |

As is evident from the results shown in the above table, injecting the tissue filler, alone, does not result in any stimulation of GAG synthesis, while injecting the peptide copper complex, either alone or in combination with the tissue filler, does result in an increase in GAG synthesis.

### Example 2

### Stimulation of Collagen and GAG Synthesis by Injection of Various Peptide Copper Complexes

The stimulation of collagen and GAG synthesis by injection of various peptide copper complexes has been determined by methods described in Example 1. The peptide copper complexes used were L-alanyl-L-histidyl-L-lysine:copper(II) (“AHK-Cu”), prepared at a molar ratio of 1 mole of peptide to one mole of copper(II), and glycyl-L-histidyl-L-lysyl-L-valyl-L-phenylalanyl-L-valine:copper(II) (“GHKVFV-Cu”), prepared at a molar ratio of 2 moles of peptide to one mole of copper(II). The peptide copper complexes were dissolved in a saline solution at a concentration of 10 milligrams/milliliter. A dose of 2.4 micromoles of peptide copper complex was injected on each treatment day.

The results of injecting the above peptide copper complexes (AHK-Cu and GHKVFV-Cu) re stimulating collagen formation, for 3 groups of rats, are shown in the table below.

| | | Mg HYP/Chamber |
|---|---|---|
| Group | Treatment | Mean ± SEM |
| 1 | Saline | 1526 ± 130 |
| 2 | AHK-Cu | 3418 ± 289 |
| 3 | GHKVFV-Cu | 4087 ± 299 |

As is evident from the results shown in the above table, the injection of the peptide copper complexes result in an increase in collagen synthesis.

The results of injecting the above peptide copper complexes (AHK-Cu and GHKVFV-Cu) re stimulating GAG (as UA) formation, for 3 groups of rats, are shown in the table below.

| | | μg Uronic Acid/Chamber |
|---|---|---|
| Group | Treatment | Mean ± SEM |
| 1 | Saline | 60 ± 5 |
| 2 | AHK-Cu | 105 ± 21 |
| 3 | GHKVFV-Cu | 79 ± 9 |

As is evident from the results shown in the above table, the injection of the peptide copper complexes results in an increase in GAG (as UA) synthesis.

### Example 3

### Stimulation of Collagen Synthesis by Injection of Various Peptide Copper Complexes Containing Leucine

The stimulation of collagen synthesis by injection of various peptide copper complexes has been determined by methods described in Examples 1 and 2. The peptide copper complexes used were glycyl-L-histidyl-L-leucine:copper(II) (“GHL-Cu”), prepared at a molar ratio of 2 moles of peptide to one mole of copper(II), and glycyl-L-histidyl-L-leucine methyl ester:copper(II) (“GHL-Me-Cu”), prepared at a molar ratio of 2 moles of peptide to one mole of copper(II). The peptide copper complexes were dissolved in a saline solution at a concentration of 10 milligrams/milliliter. A dose of 0.6 mg of peptide copper complex was injected on each treatment day.

The results of injecting the above peptide copper complexes (GHL-Cu and GHL-Me-Cu) re stimulating collagen formation, for 3 groups of rats, are shown in the table below.

| | | μg HYP/Chamber |
|---|---|---|
| Group | Treatment | Mean ± SEM |
| 1 | Saline | 1838 ± 636 |
| 2 | GHL-Cu | 3619 ± 754 |
| 3 | GHL-Me-Cu | 3357 ± 863 |

As is evident from the results shown in the above table, the injection of the peptide copper complexes result in an increase in collagen synthesis.

From the foregoing, it will be appreciated that, although specific embodiments of the invention have been described herein for purposes of illustration, various modifications may be made without deviating from the spirit and scope of the invention. Accordingly, the invention is not limited, except as by the appended claims.

What is claimed is:

1. A composition comprising a soft tissue filler and a peptide copper complex.

2. The composition of claim 1 wherein the soft tissue filler comprises a natural material derived from animal tissue.

3. The composition of claim 2 wherein the natural material is collagen, autologous fat, hyaluronic acid, a modified form of hyaluronic acid, or a mixture thereof.

4. The composition of claim 1 wherein the soft tissue filler comprises a synthetic material.

5. The composition of claim 4 wherein the synthetic material is a low melting point paraffin, a vegetable oil, lanolin, beeswax, a silicon polymer, expanded polyfluoroethylene (Teflon®), polylactic acid, polyglutamic acid, a cellulose polymer, polymethyl methacrylate, a polymer based on polymethyl methacrylate, or a mixture thereof.

6. The composition of claim 1 wherein the soft tissue filler is present at a concentration ranging from about 0.001% to about 99% by weight of the composition.

7. The composition of claim 1 wherein the soft tissue filler is present at a concentration ranging from about 0.01% to about 90% by weight of the composition.

8. The composition of claim 1 wherein the soft tissue filler is present at a concentration ranging from about 0.01% to about 50% by weight of the composition.

9. The composition of claim 1 wherein the peptide copper complex is alanyl-histidyl-lysine:copper(II).

10. The composition of claim 1 wherein the peptide copper complex is valyl-histidyl-lysine:copper(II).

11. The composition of claim 1 wherein the peptide copper complex is glycyl-histidyl-lysine:copper(II).

12. The composition of claim 1 wherein the peptide copper complex is L-alanyl-L-histidyl-L-lysine:copper(II), L-valyl-L-histidyl-L-lysine:copper(II) or glycyl-L-histidyl-L-lysine:copper(II).

13. The composition of claim 1 wherein the peptide copper complex is [glycyl-histidyl-lysine-R]:copper(II), wherein R is an alkyl moiety containing from one to eighteen carbon atoms, an aryl moiety containing from six to twelve carbon atoms, an alkoxy moiety containing from one to twelve carbon atoms, or an aryloxy moiety containing from six to twelve carbon atoms

14. The composition of claim 1 wherein the molar ratio of peptide to copper in the peptide copper complex ranges from about 1:1 to about 3:1.

15. The composition of claim 1 wherein the molar ratio of peptide to copper in the peptide copper complex ranges from about 1:1 to about 2:1.

16. The composition of claim 1 wherein the peptide copper complex is present at a concentration ranging from about 0.01% to about 10% by weight of the composition.

17. The composition of claim 1 wherein the peptide copper complex is present at a concentration ranging from about 0.025% to about 1% by weight of the composition.

18. The composition of claim 1 wherein the peptide copper complex is present at a concentration ranging from about 0.05% to about 0.5% by weight of the composition.

19. The composition of claim 1 wherein the soft tissue filler and peptide copper complex, in combination, or the peptide copper complex, is encapsulated in a liposome or microsponge adapted to aid in the delivery of the peptide copper complex, or to enhance the stability of the composition.

20. The composition of claim 1 wherein the composition is in the form of solution, thick solution, suspension, or gel.

21. The composition of claim 1, further comprising an inert and physiologically-acceptable carrier or diluent.

22. The composition of claim 21 wherein the inert and physiologically-acceptable carrier or diluent is saline or purified water.

23. The composition of claim 1, further comprising an excipient.

24. The composition of claim 23 wherein the excipient is phosphate buffered saline, bacteriostatic saline, propylene glycol, starch, sucrose, sorbitol, or a mixture thereof.

25. The composition of claim 1, further comprising a thickening agent.

26. The composition of claim 25 wherein the thickening agent is acrylamides copolymer, carbomer, hydroxyethylcellulose, hydroxypropylcellulose, polyacrylic acid, polymethacrylic acid, polyvinyl alcohol, or a mixture thereof.

27. The composition of claim 1, further comprising an emulsifying agent.

28. The composition of claim 27 wherein the emulsifying agent is caprylic/capric triglyceride, ceteareth-7, cetyl alcohol, cetyl phosphate, isosteareth-11, sodium isostearate, or a mixture thereof.

29. The composition of claim 1, further comprising a preservative.

30. The composition of claim 29 wherein the preservative is benzyl alcohol, a paraben, diazolidinyl urea, DMDM hydantoin, phenoxyethanol, iodopropynyl butylcarbamate, or a mixture thereof.

31. A method for treating skin defects comprising injecting into an area of skin in need thereof an effective amount of the composition of claim 1.

32. A method for treating skin defects comprising injecting into an area of skin in need thereof an effective amount of a soft tissue filler, followed by injecting into the area an effective amount of a composition comprising peptide copper complex.

33. A method for treating skin defects comprising injecting into an area of skin in need thereof an effective amount of a soft tissue filler, followed by topically applying an effective amount of a composition comprising peptide copper complex.

34. The method of claim 33 wherein the composition comprising peptide copper complex, further comprises a sunscreen agent, a skin lightening agent, a tanning agent, a skin conditioning agent, a skin protectant, an emollient, a humectant, or a mixture thereof.
